(19) United States
(12) Patent Application Publication
(10) Pub. No.: US 2009/0053755 A1
(43) Pub. Date: Feb. 26, 2009

(54) PROBE BASED MOLECULAR SIGNAL DELIVERY FOR PRECISE CONTROL AND MEASUREMENT OF SINGLE CELL RESPONSES

(76) Inventors: Todd Aaron Sulchek, Oakland, CA (US); Amy Lynn Hiddessen, Dublin, CA (US)

Correspondence Address: Lawrence Livermore National Security, LLC, LAWRENCE LIVERMORE NATIONAL LABORATORY, PO BOX 808, L-703, LIVERMORE, CA, 94551-0808, US

(21) Appl. No.: 12/125,800

(22) Filed: May 22, 2008

Related U.S. Application Data
(60) Provisional application No. 60/931,893, filed on May 24, 2007.

(51) Int. Cl. C12Q 1/02; C12M 1/34

(52) U.S. Cl. 435/29; 435/288.7

(57) ABSTRACT

A system for producing and visualizing the response of a cell. The cell has a surface. A probe is provided. The probe is derivitized so that at least one bio-active molecule is covalently linked to the probe. The probe with the at least one bio-active molecule covalently linked to the probe is positioning over the surface of the cell. The at least one bio-active molecule is delivered to the surface of the cell producing a response of the cell. The response of the cell to the at least one bio-active molecule is visualized.

### CROSS-REFERENCE TO RELATED APPLICATIONS

This application claims the benefit of U.S. Provisional Patent Application No. 60/931,893 filed May 24, 2007 and titled “Probe Based Molecular Signal Delivery for Precise Control and Measurement of Single Cell Responses.” U.S. Provisional Patent Application No. 60/931,893 filed May 24, 2007 and titled “Probe Based Molecular Signal Delivery for Precise Control and Measurement of Single Cell Responses” is incorporated herein by this reference.

The United States Government has rights in this invention pursuant to Contract No. DE-AC52-07NA27344 between the United States Department of Energy and Lawrence Livermore National Security, LLC for the operation of Lawrence Livermore National Laboratory.

### BACKGROUND

1. Field of Endeavor

The present invention relates to cell response and more particularly to probe based molecular signal delivery for precise control and measurement of single cell responses.

2. State of Technology

United States Published Patent Application No. 2003/0228695 by Chikashi Nakamura et al for a cell-manipulating apparatus and method of using the same provides the following state of technology information: “Heretofore, there have been known various conventional techniques for introducing a gene into a cell and expressing the gene, such as a physical technique including an electroporation method, a particle gun method, and a microinjection method; a technique utilizing cell-endocytosis including a calcium phosphate method, a DEAE-dextran method, and a lipofection method; and another technique including an infection method using virus vectors, and a liposome method of introducing a liposome-encapsulated gene into a cell through cell fusion. All of these gene-introducing techniques focus only on allowing the introduced gene to be permanently held in the cell but not on allowing temporal dynamic changes appearing in the cell to be observed in detail, for example, from the gene introduction to gene expression.”

U.S. Pat. No. 6,758,961 for positioning and electrophysiological characterization of individual cells and reconstituted membrane systems on microstructured carriers issued to Horst Vogel provides the following state of technology information: “Many biologically important signal transduction processes, such as nerve conduction, occur on or in the cell membranes. Therefore, it is not surprising that the biological functions of membrane proteins in general and of neuroreceptors in particular are influenced by pharmacologically active compounds (J.-P. Changeux (1993), “Chemical signalling in the brain”, Sci. Am. November, pages 30 and following; A. G. Gilman (1995), Angew. Chem. Int. Ed. Engl. 34:1406-1428; M. Rodbell (1995), Angew. Chem. Int. Ed. Engl. 34: 1420-1428). The functional understanding of the molecular interactions on receptors, as well as the use of receptors in the screening of active compounds, play a central role in modern drug development.”

### SUMMARY

Features and advantages of the present invention will become apparent from the following description. Applicants are providing this description, which includes drawings and examples of specific embodiments, to give a broad representation of the invention. Various changes and modifications within the spirit and scope of the invention will become apparent to those skilled in the art from this description and by practice of the invention. The scope of the invention is not intended to be limited to the particular forms disclosed and the invention covers all modifications, equivalents, and alternatives falling within the spirit and scope of the invention as defined by the claims.

The present invention provides a system for producing and visualizing the response of a cell. The cell has a surface. A probe is provided. The probe is derivitized so that at least one bio-active molecule is covalently linked to the probe. The probe with at least one bio-active molecule covalently linked to the probe is positioning over the surface of the cell. At least one bio-active molecule is delivered to the surface of the cell producing a response by the cell. The response of the cell to at least one bio-active molecule is visualized or quantified. In one embodiment the probe is the tip of an atomic force microscope cantilever. In another embodiment the probe is a bead on the end of an aspirated pipette.

One embodiment of the present invention provides a method of producing and visualizing the response of a cell, wherein the cell has a surface, including the steps of providing a probe with a tip surface, derivitizing the probe so that at least one reactive chemical group is covalently linked to the tip surface of the probe, positioning the tip surface of the probe with the at least one reactive chemical group covalently linked to the tip surface over the surface of the individual cell, delivering the at least one reactive chemical group to the surface of the cell producing a response of the cell, and visualizing the response of the cell to the at least one reactive chemical group.

One embodiment of the present invention provides an apparatus for delivering a molecule or a signal to the surface of an individual cell and visualizing the response of the cell, wherein the individual cell has a surface and includes at least one target surface receptor on the surface of the cell, and wherein the cell includes at least one up stream intercellular signaling transducer, and at least one down stream intercellular signaling transducer. The apparatus includes a functionalized probe; at least one signal molecule operatively connected to the functionalized probe, wherein the probe and at least one signal molecule is adapted to be delivered to at least one target surface receptor on the surface of the cell; and a system for visualizing the response of the cell, wherein at least one up stream intercellular signaling transducer and at least one down stream intercellular signaling transducer interact with at least one signal molecule.

The invention is susceptible to modifications and alternative forms. Specific embodiments are shown by way of example. It is to be understood that the invention is not limited to the particular forms disclosed. The invention covers all modifications, equivalents, and alternatives falling within the spirit and scope of the invention as defined by the claims.

### BRIEF DESCRIPTION OF THE DRAWINGS

The accompanying drawings, which are incorporated into and constitute a part of the specification, illustrate specific embodiments of the invention and, together with the general description of the invention given above, and the detailed description of the specific embodiments, serve to explain the principles of the invention.

FIG. 1 illustrates one embodiment of the system for probe-based signal delivery to individual cells.

FIG. 2 illustrates additional details of the system for probe-based signal delivery to individual cells shown in FIG. 1.

FIGS. 3A and 3B illustrate additional details of the system for probe-based signal delivery to individual cells shown in FIG. 1.

FIG. 4 illustrates the response of the cell.

FIG. 5 illustrates another embodiment of the system for probe-based signal delivery to individual cells.

### DETAILED DESCRIPTION OF THE INVENTION

Referring to the drawings, to the following detailed description, and to incorporated materials, detailed information about the invention is provided including the description of specific embodiments. The detailed description serves to explain the principles of the invention. The invention is susceptible to modifications and alternative forms. The invention is not limited to the particular forms disclosed. The invention covers all modifications, equivalents, and alternatives falling within the spirit and scope of the invention as defined by the claims.

It is important to understand how an organism's constituent cells respond to molecules and signals derived from environmental, pharmaceutical, and pathogenic sources. It is the individual cell response to these externally derived signals that determines whether the cell or tissue dies, whether the drug heals, or whether the microbe infects. Understanding and controlling the cell response to external factors is a primary goal of human health research.

Cell response to be a chemical, biological, physical or informational product of a cell that directly results from single or multiple stimuli are defined. Conventional methods to study the response of cells, either individually, as part of a tissue, or in a whole organism, rely upon observing or measuring the effect of the stimuli upon many cells simultaneously. Measurements of response of many cells to a signal input, termed ensemble measurements, are traditionally used simply because it is simpler to operate with macroscopic volumes of signal and cellular material and the response of a large number of cells is typically easier to detect.

Ensemble-based methods for cell signal delivery suffer from a critical problem: the measured response is an average of all the cells in the measurement volume. Therefore, variations in the response of individual cells due to cell type, internal cell state, duration of contact with signal, or spatial location of signal binding will not be resolvable. Resolving the variation in response of individual cells is critical, however, as this variation is a primary factor in determining how consistent a given cell will respond to a pharmaceutical or environmental signal. Thus, conventional assays suffer from fundamental limitations.

Current technology provides a variety of biochemical and biophysical methods for facilitating read-out and measurement of the response of a cell to imposed external signals. Reporter assays and cellular labeling methods, such as luminescence or fluorescence based approaches, are commonly used to facilitate detection and/or tracking of proteomic or genomic concentration and localization within cells. These and other approaches are often combined with optical microscopy, fluorescent microscopy, force spectroscopy, flow cytometry, mRNA analysis, mass spectroscopy or Raman spectroscopy to detect and analyze changes in cell state.

Moreover, the stimulus applied to a cell is typically added in an imprecise manner, for example through pipette of a volume many times the size of single cells. This limits the accuracy with which the response to the stimuli may be studied. A variety of approaches exist for delivering external stimuli to cells. These methods are typically applied with one or more biochemical, optical or spectroscopic methods for subsequent analyses of cell response. A conventional ensemble assay for delivering signal molecules to cells and measuring cell response is the pipetting of signals to cells plated in a multiwell plate or Petri dish, an approach commonly used in biology. Beads derivatized with specific signal molecules such as antibodies have been used to both deliver stimuli to and activate cells. Substrates engineered with patterned arrays of signal molecules provide an alternative method for observing the effects of cell contact to a particular substance. Recent developments in microfluidics have also established new methods for delivering signal molecules to cells via channels engineered to flow small reagent volumes to cells. However, a method that reliably and consistently delivers an externally derived signal of variable strength with temporal and spatial precision to a single, specified cell is still lacking.

Previously described methods for delivering signals to individual cells do not provide the user-defined molecular scale control to the delivery. For example, viral targeting, signal-modified cell targeting, and antibody targeting can deliver an agent to cells, but do not provide a method to control the number of signal molecules that contact the cell, the duration of contact, or the location of contact with the cell.

Applicants' method uses a microscopic probe, such as an actuated polystyrene bead or atomic force microscope (AFM) cantilever tip, to initiate a chemical, biological, or physical signal upon the surface of a single cell. A response in the target cell can be then correlated to the initiated signal. The cell response can be read out using a variety of biochemical and biophysical techniques. The delivered signal can be varied; examples of signals include pressure, heat, metabolite, protein, radioisotope, pharmaceutical drug candidate or any other molecule that affects the internal function or state of the cell. The measured response can include a change in morphology, metabolism, gene expression, or production or reorganization of specialized proteins.

A user-controlled microscopic probe upon which Applicants confine the desired signal was used. The probe can be modified to define the parameters of signal contact to the individual cell surface. These parameters include molecular number, density, time and duration of contact, and spatial location. The ideal signal delivery platform requires: 1) a delivery probe of a similar size to that of the signal molecule, 2) nanometer spatial control in three dimensions and millisecond time response, and 3) minimal unintended, probe-induced response. A preferred embodiment of the platform to control a microscopic probe is the atomic force microscope (AFM).

This method does not preclude the ability of delivering multiple signals to more than one cell. In fact, parallel, high throughput methods for control of AFM probes have been demonstrated. Thus, the combination of probe arrays with cell arrays in a single platform, a key aspect of this invention, opens up the possibility of studying the individual response of many cells to a multitude of signal types. The control of signal inputs combined with methods to measure the response will be an essential advancement in quantification of biological processes.

Small quantities of molecules and signals are delivered to the surface of individual cells in a temporally, spatially and numerically controlled manner. Applicants' method can directly deliver both physical and chemical signals to cells. Applicants deliver the cellular signals via the surface of a small probe, for example the tip of an atomic force microscope cantilever or a bead on the end of an aspirated pipette, that can be controlled with an external actuator. In the case of chemical signals, the probe can be functionalized with the molecule or organism of choice at a controlled concentration. Applicants can maneuver the probe so that it touches the cell surface and sequentially initiates a cell response to the impinging signal. Because the user controls the delivery of the signal proteins, metabolites, or other molecular and physical stimuli to the cell surface, the cellular response can be correlated to the input signal with high temporal, spatial, and numerical precision. The cell response can be visualized and quantified using a variety of biochemical and biophysical techniques. To enhance read-out quality, the cell can be bioengineered to provide an improved response. The probe and cells can be arrayed to permit simultaneous monitoring of the responses of many cell types to different signal molecules; hence, Applicants method supports high throughput and multiplexed applications.

Applicants' invention has many uses. The control of signal input to individual cells will enable a variety of pharmaceutical, medical and biosecurity discoveries and applications.

First, Applicants' method allows the study of the effect on individual cells of any drug that targets a cell surface receptor, which accounts for over 50% of drug targets. This response can be monitored using a variety of methods described in section VIII. Study of individual cells is important to drug development because it provides an avenue to study why some drugs affect individuals in different ways and why drugs affect various cell types in different ways. The method enables study of the dynamics of the drug's effect, improves Applicants ability to determine proper dosage, and is compatible with highly parallel measurements of various drug targets under identical conditions.

Second, Applicants' method provides a direct method to study the process of stem cell fate decisions by precisely controlling the various biochemical factors that direct the cell's choice to proliferate or differentiate. Applicants' method allows control of local concentration of signal, duration of signal contact, and spatial location. Applicants' method provides a method to reliably control the physical and chemical stimuli that affect stem cell differentiation and allows both mechanical and biochemical factors to be tested individually or simultaneously. Applicants' method can be applied to precisely determine the specific roles of known biochemical factors identified through more conventional molecular genetic approaches, and can also be applied to discover new molecular factors that direct stem cell behavior.

Third, Applicants' method provides a high throughput method to identify potential pathogen virulence factors and determine their response in a host. The method is applicable to both a research and clinical setting. Unknown pathogen candidates can be immobilized on the probe surface as intact organisms or as purified virulence factors. The probe surface can then be tested against individual host cells to determine their effect upon host cell, including indication of virulence. The probe can be functionalized with varying concentrations of known/potential virulence factors, such as invasin proteins, to determine concentration dependence of virulence. As the host cell response is the truest test of virulence, this method can surpass genomic and proteomic signature typing methods for determining pathogenicity, particularly since many pathogens are constantly evolving at the molecular level to overcome host defenses.

Fourth, Applicants' method is applicable to a variety of other uses where it is important to precisely deliver single to multiple molecules and structures containing them. Applicable chemical and biological stimuli include, but are not limited to: proteins, DNA, PNA, RNA, antibodies, fatty-acids, drug compounds, radioisotopes, polymeric molecules and structures (branched or linear) including dendrimers, amphiphilic molecules, particles (e.g., micro- and nanoparticles of metallic, semiconductor, and polymeric origin, which may be surface functionalized with specific molecular groups), single to multiple biological organisms, including cells (eukaryotic and prokaryotic) and pathogens (bacteria, viruses, fungi). Applicable physical stimuli include, but are not limited to: heat, mechanical force, dynamic pressure, electromagnetic fields, radiation, acoustic stimulation and near-field/localized optical stimulation.

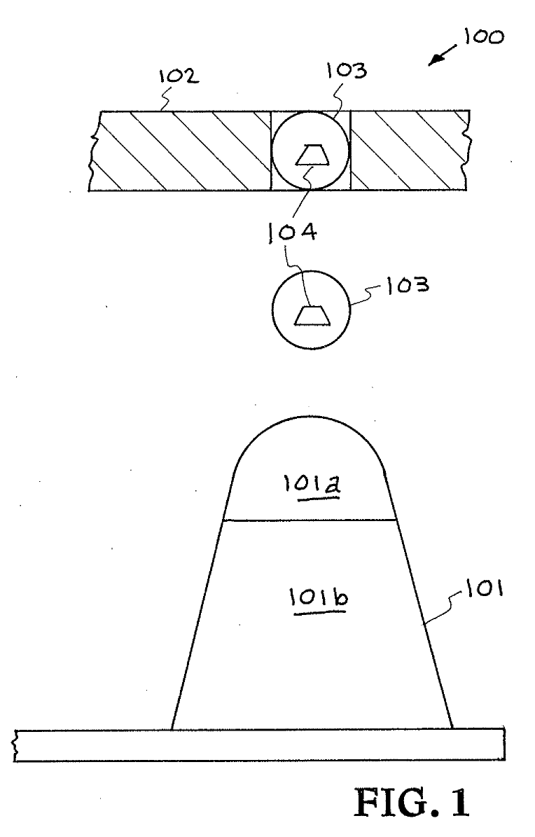

Referring now to the drawings and in particular to FIGS. 1, 2, 3A, and 3B, one embodiment of a system for probe based signal delivery to individual cells is illustrated. The system is designated generally by the reference numeral 100. The system 100 provides a system for visualizing the response of a cell 105. The cell 105 has a surface 106. The system 100 includes providing a probe 101, derivitizing the probe 101 so that a bio-active molecule 104 is covalently linked to the probe 101. The probe with said at least one bio-active molecule 104 covalently linked to the probe 101 is positioned over the surface of the cell 105. The bio-active molecule 104 is delivered to the surface of the cell 105 producing a response of the cell 105. The response of the cell 105 to said at least one bio-active molecule 104 is visualized 301.

Referring now to FIG. 1 details of one embodiment of the system 100 for probe-based signal delivery to individual cells is illustrated. The system 100 illustrated in FIG. 1 includes the following structural elements: a functionalized probe 101 having a body 101a and a tip 101b, a fluid dispenser 102, a solution 103, and a signal molecule 104.

The functionalized probe 101 can be the tip of an atomic force microscope cantilever. The functionalized probe 101 can also be a bead on the end of an aspirated pipette. The functionalized probe 101 has a body 101b and a tip 101b. The tip 101b can have silica, gold, or other reactive material on the tip 101b of the probe 101.

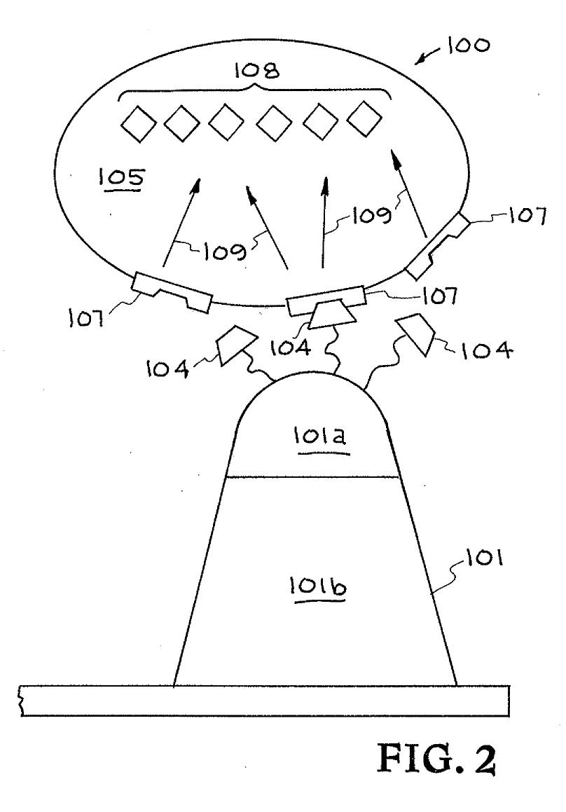

Referring now to FIG. 2 additional details of one embodiment of the system 100 for probe-based signal delivery to individual cells is illustrated. The system 100 illustrated in FIG. 2 includes the following elements: cell 105, cell surface 106, target surface receptors 107, and intercellular signaling transducer 108.

As illustrated in FIG. 2, the probe 101 is functionalized by derivitizing the probe tip 101a by covalently linking the reactive chemical group 104 to the probe tip 101a. As illustrated in FIGS. 1 and 2 the probe 101 is functionalized by derivitizing the probe tip 101a by delivering the microscopic droplet 101 containing the bio-active molecule 104 to the probe 101. The microscopic droplet 103 containing the bio-active molecule 104 is delivered to the probe 101 through the nozzle 102. The step of derivitizing the probe 101 is accomplished by delivering an incubated solution 103 of the biomolecule 104 to the probe 101. The step of derivitizing the probe 101 is accomplished by adhering the biomolecule 104 to the probe 101 through reactive chemistries. In one embodiment the step of derivitizing the probe 101 is accomplished by adhering the biomolecule 104 to the probe 101 through 1-Ethyl-3-[3-dimethylaminopropyl]carbodiimide hydrochloride reactive chemistries. In one embodiment of the present invention the probe 101 is functionalized by derivitizing the probe tip 101a by covalently linking an amine reactive chemical group to the probe 101.

As illustrated in FIG. 2, the bio-active molecule 104 that has been covalently linked to the probe 101 is positioned over the surface 106 of the cell 105. This is accomplished by positioning the probe 101 with the bio-active molecule 104 covalently linked to the probe 101 over the surface 106 of the cell 105. This may be accomplished in one embodiment by the bio-active molecule 104 that is covalently linked to the probe 101 being positioned over the surface 106 of a live cell 105. This may be accomplished in one embodiment by the bio active molecule 104 that is covalently linked to the probe 101 being positioned over tissue. In another embodiment this is accomplished by the bio-active molecule 104 that is covalently linked to the probe 101 being positioned over a cell mimetic.

The bio-active molecules 104 are delivered to the surface 106 of the cell 105 producing a response of the cell 105. The bio-active molecules 104 are directed to the target surface receptors 107. This produces a response of the cell 105 through the intercellular signaling transducer 108 as indicated by the arrows 109.

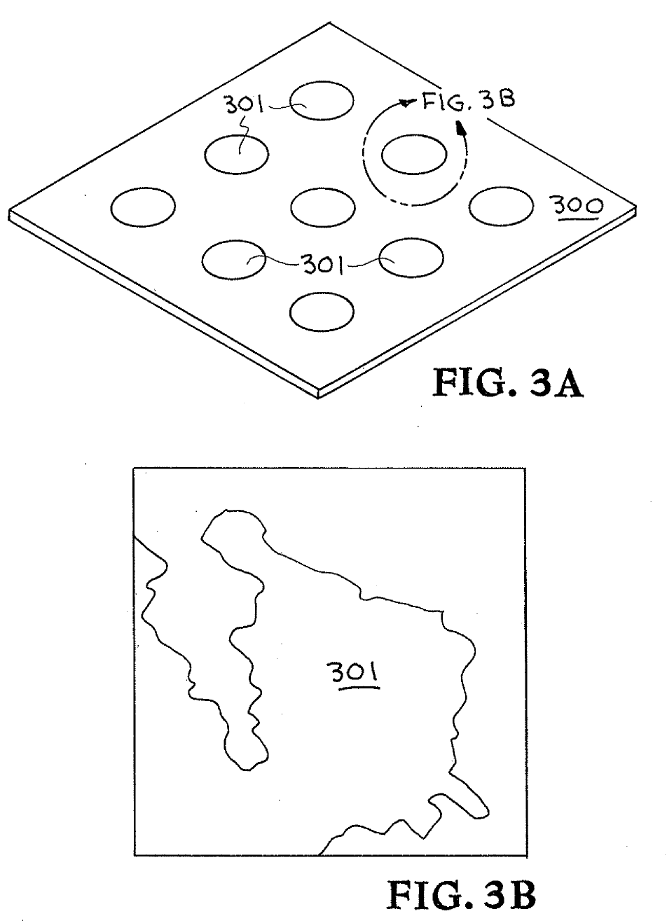

Referring now to FIGS. 3A and 3B, the bio-active molecule 104 has been delivered to the surface of the cell producing a response of the cell. The response of the cell 105 to the bio-active molecule 104 is visualized as illustrated at 301. A cell array 300 illustrates cellular response read-out 301. The response of the cell 105 to the bio-active molecule 104 can be visualized by measuring the change of the cell 105. In one embodiment the response of the cell 105 to the bio-active molecule 104 can be visualized by measuring the change of phosphorolation states of the cell 104. In another embodiment the response of the cell 105 to the bio-active molecule 104 can be visualized by measuring the change of phosphorolation states of the cell 104. In one embodiment the response of the cell 105 to the bio-active molecule 104 can be visualized by visualizing the response of the cell 105 using ionic current measurement. In one embodiment the response of the cell 105 to the bio-active molecule 104 can be visualized by visualizing the response of the cell 105 using spectroscopy. In one embodiment the response of the cell 105 to the bio-active molecule 104 can be visualized by visualizing the response of the cell 105 using stiffness measurement. In one embodiment the response of the cell 105 to the bio-active molecule 104 can be visualized by visualizing the response of the cell 105 using fluorescence detection. In one embodiment the response of the cell 105 to the bio-active molecule 104 can be visualized by using a pharmacological pathway that is fluorescently labeled with a reporter whereupon activation of the pathway a fluorescent signal is released and observed with optical microscopy.

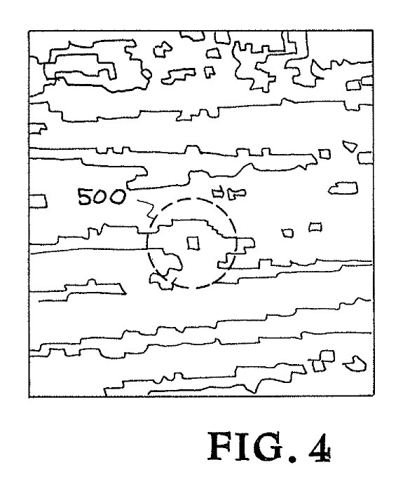

Referring now to FIG. 4 visualization of the response of the cell 105 to the bio-active molecule is illustrated. The bio-active molecule has been delivered to the surface of the cell producing a response of the cell 105. The response of the cell is show within the circle 500. The response 500 of the cell 105 to the bio-active molecule can be visualized by the change of the cell 105. The change of the cell 105 can be monitored by optical microscopy, fluorescent microscopy, force spectroscopy, flow cytometry, mRNA analysis, mass spectroscopy or Raman spectroscopy to detect and analyze changes in cell state.

The change of the cell 105 can be monitored in various embodiments. In one embodiment the response 500 of the cell 105 to the bio-active molecule can be visualized by measuring the change of phosphorolation states of the cell 104. In another embodiment the response 500 of the cell 105 to the bio-active molecule can be visualized by measuring the change of phosphorolation states of the cell 104. In one embodiment the response 500 of the cell 105 to the bio-active molecule can be visualized by visualizing the response of the cell 105 using ionic current measurement. In one embodiment the response of the cell to the bio-active molecule can be visualized by visualizing the response of the cell using spectroscopy. In one embodiment the response of the cell to the bio-active molecule can be visualized by visualizing the response of the cell using stiffness measurement. In one embodiment the response of the cell to the bio-active molecule can be visualized by visualizing the response of the cell using fluorescence detection. In one embodiment the response of the cell to the bio-active molecule can be visualized by using a pharmacological pathway that is fluorescently labeled with a reporter whereupon activation of the pathway a fluorescent signal is released and observed with optical microscopy.

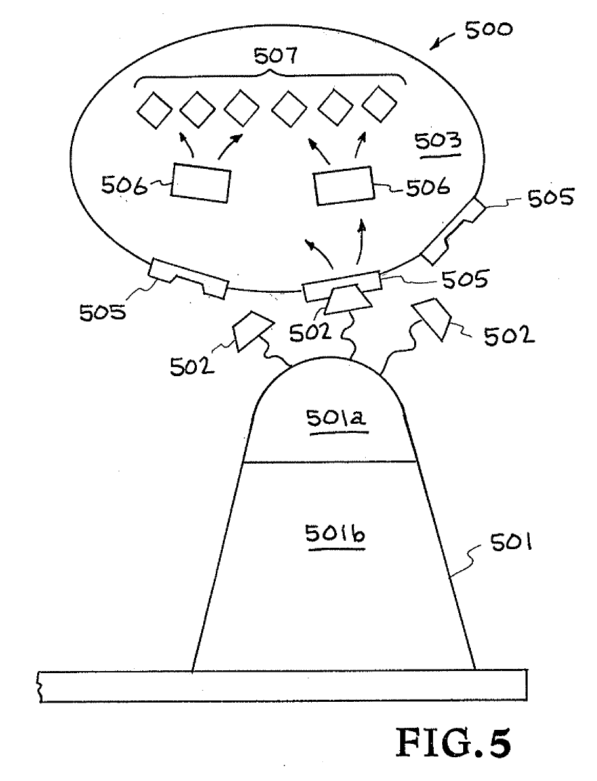

Referring now to FIG. 5 another embodiment of a system for probe-based signal delivery to individual cells is illustrated. The system is designated generally by the reference numeral 500. The system 500 illustrated in FIG. 5 includes the following elements: probe 501 having body 501b and probe tip 501a, biomolecules 502, cell 503, cell surface 504, target surface receptors 505, an intercellular signaling transducer (up stream) 506, and intercellular signaling transducer (down stream) 507.

As illustrated in FIG. 5, the probe 501 is functionalized by derivitizing the probe tip 501a by covalently linking the reactive biomolecules 502 to the probe tip 501a. The step of derivitizing the probe 501 is accomplished by adhering the biomolecules 502 to the probe 501 through reactive chemistries. In one embodiment the step of derivitizing the probe 501 is accomplished by adhering the biomolecules 502 to the probe 501 through 1-Ethyl-3-[3-dimethylaminopropyl]carbodiimide hydrochloride reactive chemistries. In one embodiment of the present invention the probe 501 is functionalized by derivitizing the probe tip 501a by covalently linking an anamine reactive chemical group to the probe 501.

As illustrated in FIG. 5, the bio-active molecule 502 that has been covalently linked to the probe 501 is positioned over the surface 504 of the cell 503. This is accomplished by positioning the probe 501 with the bio-active molecule 502 covalently linked to the probe 501 over the surface 504 of the cell 503. This may be accomplished in one embodiment by the bio-active molecule 502 that is covalently linked to the probe 501 being positioned over the surface 504 of a live cell 503. This may be accomplished in one embodiment by the bio-active molecule 502 that is covalently linked to the probe 501 being positioned over tissue. In another embodiment this is accomplished by the bio-active molecule 502 that is covalently linked to the probe 501 being positioned over a cell mimetic.

The bio-active molecules 504 are delivered to the surface 504 of the cell 503 producing a response of the cell 503. The bio-active molecules 502 are directed to the target surface receptors 505. This produces a response of the cell 503 through the intercellular signaling transducer 506 and the intercellular signaling transducer 507. The response of the cell 503 to the bio-active molecule 502 is then visualized. The response of the cell to the bio-active molecule can be visualized by measuring the change of the cell. In one embodiment the response of the cell to the bio-active molecule can be visualized by measuring the change of phosphorolation states of the cell. In another embodiment the response of the cell to the bio-active molecule can be visualized by measuring the change the change of phosphorolation states of the cell. In one embodiment the response of the cell to the bio-active molecule can be visualized by visualizing the response of the cell using ionic current measurement. In one embodiment the response of the cell to the bio-active molecule can be visualized by visualizing the response of the cell using spectroscopy. In one embodiment the response of the cell to the bio-active molecule can be visualized by visualizing the response of the cell using stiffness measurement. In one embodiment the response of the cell to the bio-active molecule can be visualized by visualizing the response of the cell using fluorescence detection. In one embodiment the response of the cell to the bio-active molecule can be visualized by using a pharmacological pathway that is fluorescently labeled with a reporter whereupon activation of the pathway a fluorescent signal is released and observed with optical microscopy.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

The invention claimed is:

1. A method of producing and visualizing the response of a cell, wherein the cell has a surface, comprising the steps of:
providing a probe,
derivitizing said probe so that at least one bio-active molecule is covalently linked to said probe,
positioning said probe with said at least one bio-active molecule covalently linked to said probe over the surface of the cell,
delivering said at least one bio-active molecule to the surface of the cell producing a response of the cell, and
visualizing said response of the cell to said at least one bio-active molecule.

2. The method of producing and visualizing the response of a cell of claim 1 wherein said step of providing a probe comprises providing a probe that is the tip of an atomic force microscope cantilever.

3. The method of producing and visualizing the response of a cell of claim 1 wherein said step of providing a probe comprises providing a probe that is a bead on the end of an aspirated pipette.

4. The method of producing and visualizing the response of a cell of claim 1 wherein said step of providing a probe comprises providing a probe with silica, gold, or other reactive material on said probe.

5. The method of producing and visualizing the response of a cell of claim 1 wherein said step of derivitizing said probe includes covalently linking a reactive chemical group to said probe.

6. The method of producing and visualizing the response of a cell of claim 1 wherein said step of derivitizing said probe includes covalently linking amine reactive chemical group to said probe.

7. The method of producing and visualizing the response of a cell of claim 1 wherein said step of derivitizing said probe comprises delivered a microscopic droplet containing the bio-active molecule to said probe.

8. The method of producing and visualizing the response of a cell of claim 1 wherein said step of derivitizing said probe comprises delivered a microscopic droplet containing the bio-active molecule to said probe through a nozzle to said probe.

9. The method of producing and visualizing the response of a cell of claim 1 wherein said step of derivitizing said probe comprises delivered an incubated solution of the biomolecule to said probe.

10. The method of producing and visualizing the response of a cell of claim 1 wherein said step of derivitizing said probe comprises adhering the biomolecule to said probe through reactive chemistries.

11. The method of producing and visualizing the response of a cell of claim 1 wherein said step of derivitizing said probe comprises adhering the biomolecule to said probe through 1-Ethyl-3-[3-dimethylaminopropyl]carbodiimide hydrochloride reactive chemistries.

12. The method of producing and visualizing the response of a cell of claim 1 wherein said step of positioning said probe with said at least one bio-active molecule covalently linked to said probe over the surface of the cell comprises positioning said probe with said at least one bio-active molecule covalently linked to said probe over the surface of a live cell.

13. The method of producing and visualizing the response of a cell of claim 1 wherein said step of positioning said probe with said at least one bio-active molecule covalently linked to said probe over the surface of the cell comprises positioning said probe with said at least one bio-active molecule covalently linked to said probe over the surface of tissue.

14. The method of producing and visualizing the response of a cell of claim 1 wherein said step of positioning said probe with said at least one bio active molecule covalently linked to said probe over the surface of the cell comprises positioning said probe with said at least one bio-active molecule covalently linked to said probe over the surface of a cell mimetic.

15. The method of producing and visualizing the response of a cell of claim 1 wherein said step of visualizing said response of the cell to said at least one bio-active molecule comprises measuring the change of the cell.

16. The method of producing and visualizing the response of a cell of claim 1 wherein said step of visualizing said response of the cell to said at least one bio-active molecule comprises measuring the change of phosphorolation states of the cell.

17. The method of producing and visualizing the response of a cell of claim 1 wherein said step of visualizing said response of the cell to said at least one bio-active molecule comprises measuring the change of morphology of the cell.

18. The method of producing and visualizing the response of a cell of claim 1 wherein said step of visualizing said response of the cell to said at least one bio-active molecule comprises visualizing said response of the cell using microscopy.

19. The method of producing and visualizing the response of a cell of claim 1 wherein said step of visualizing said response of the cell to said at least one bio-active molecule comprises visualizing said response of the cell using ionic current measurement.

20. The method of producing and visualizing the response of a cell of claim 1 wherein said step of visualizing said response of the cell to said at least one bio-active molecule comprises visualizing said response of the cell using spectroscopy.

21. The method of producing and visualizing the response of a cell of claim 1 wherein said step of visualizing said response of the cell to said at least one bio-active molecule comprises visualizing said response of the cell using stiffness measurement.

22. The method of producing and visualizing the response of a cell of claim 1 wherein said step of visualizing said response of the cell to said at least one bio-active molecule comprises visualizing said response of the cell using fluorescence detection.

23. The method of producing and visualizing the response of a cell of claim 1 wherein said step of visualizing said response of the cell to said at least one bio-active molecule comprises using a pharmacological pathway that is fluorescently labeled with a reporter whereupon activation of the pathway a fluorescent signal is released and observed with optical microscopy.

24. A method of producing and visualizing the response of a cell, wherein the cell has a surface, comprising the steps of:
providing a probe with a tip surface,
derivitizing said probe so that at least one reactive chemical group is covalently linked to said tip surface of said probe,
positioning said tip surface of said probe with said at least one reactive chemical group covalently linked to said tip surface over the surface of the individual cell,
delivering said at least one reactive chemical group to the surface of the cell producing a response of the cell, and
visualizing said response of the cell to said at least one reactive chemical group.

25. An apparatus for delivering a molecule or a signal to the surface of an individual cell and visualizing the response of the cell, wherein the individual cell has a surface and includes at least one target surface receptor on the surface of the cell, and wherein the cell includes at least one up stream intercellular signaling transducer, and at least one down stream intercellular signaling transducer, comprising:
a functionalized probe;
at least one signal molecule operatively connected to said functionalized probe, wherein said probe and said at least one signal molecule is adapted to be delivered to the at least one target surface receptor on the surface of the cell; and
a system for visualizing the response of the cell, wherein the at least one up stream intercellular signaling transducer and the at least one down stream intercellular signaling transducer interact with said at least one signal molecule.

26. The apparatus of claim 1 wherein said functionalized probe is the tip of an atomic force microscope cantilever.

27. The apparatus of claim 1 wherein said functionalized probe is a pipette microaspirator.
